(19) United States
(12) Patent Application Publication
(10) Pub. No.: US 2011/0064768 A1
(43) Pub. Date: Mar. 17, 2011

(54) Immunogenic Compositions

(76) Inventors: Simon Draper, Oxfordshire (GB); Adrian Hill, Oxfordshire (GB); Fergal Hill, Lyon (FR)

(21) Appl. No.: 12/595,351

(22) PCT Filed: Apr. 10, 2008
(86) PCT No.: PCT/GB2008/001271

§ 371 Date: Apr. 23, 2010

(30) Foreign Application Priority Data
Apr. 10, 2007 (GB) 0706912.3

(51) Int. Cl. A61K 39/12; C12N 15/63; A61K 39/275; A61K 39/285; A61K 39/245; A61K 39/235; A61P 33/06; A61P 31/12; A61P 31/20; A61P 31/22; A61P 31/14; A61P 37/04

(52) U.S. Cl. 424/218.1; 435/320.1; 424/204.1; 424/232.1; 424/229.1; 424/231.1; 424/230.1; 424/233.1

(57) ABSTRACT

An immunogenic composition comprising a viral vector, said vector comprising a nucleic acid sequence encoding a C4bp domain or variant or fragment thereof and a nucleic acid sequence encoding an antigen of interest.

### FIELD OF INVENTION

The present invention relates to Immunogenic viral vector compositions and vaccines comprising a C4bp domain and an antigen, and to methods of their use for providing an enhanced T cell response.

### BACKGROUND

Vaccination has proved to be one of the most effective means of preventing diseases, particularly infectious diseases. Most vaccines work by inducing antibodies that are protective against infection by the relevant pathogen. However many new vaccines target the cellular arm of the immune system and work by inducing effector and memory T cells. These can target intracellular pathogens and tumours. Many new T cell inducing vaccines that may be used either prophylactically or therapeutically are in development.

T cells induced by vaccination may be useful in various ways. As well as reducing risk of diseases in the vaccinee they may be used in adoptive transfer protocols to reduce risk of infection or disease in those receiving these cells. They may also be useful diagnostically.

An increasingly widely used method of inducing an immune response is to clone an antigen or epitope of interest into a vector. Vectors may be plasmid, bacterial or viral. Plasmid DNA vaccines are under intensive development and a variety of viral vectors appear useful for vaccination. These include poxviruses such as modified vaccinia virus Ankara (MVA), avipox vectors such as fowlpox and canarypox and ALVAC, herpesvirus vectors (including herpes simplex and CMV), alphaviruses and adenoviruses.

Diseases that might be targeted by improved vectors include but are not limited to malaria, tuberculosis, HIV/AIDS, HCV, HBV, HSV, HPV, CMV, diseases caused by encapsulated bacteria such as the pneumococcus, parasitic diseases such as leishmaniasis, and a wide range of tumours and cancers, such as lymphoma, leukaemias, melanoma, renal, breast, lung, prostate, pancreatic and colorectal cancers.

Malaria remains one of the world's greatest public health challenges. Plasmodium falciparum infection affects over 500 million people annually, resulting in the death of 1-2 million individuals^{1,2}. The development of an effective vaccine remains an important goal for the safe and cost-effective control of this disease.

Immunity to the blood-stage of malaria infection is predominantly antibody-mediated, with merozoite surface antigens, including merozoite surface protein-1 (MSP-1), amongst the targets of protective responses^{3,4}. MSP-1 is synthesised as a 190-230-kDa polypeptide which undergoes proteolytic processing during erythrocyte invasion^{s}. The 42-kDa C-terminus (MSP-1₄₂) is finally cleaved into 33-kDa and 19-kDa fragments (MSP-1₃₃ and MSP-1₁₉ respectively), of which only MSP-1₁₉ remains associated with the merozoite surface⁶. MSP-1₄₂ is a leading blood-stage malaria vaccine candidate antigen and is capable of inducing protective responses in mice and monkeys, largely dependent on the presence of high titre antibody responses against MSP-1₁₉ at the time of malaria challenge⁷⁻⁹. Antibody responses against MSP-1₃₃ are not protective^{10,11}.

The contribution of T cell responses to blood-stage immunity remains less well defined¹². In the case of MSP-1₄₂, CD4⁺ T cell epitopes have been described within MSP-1₃₃ in a number of Plasmodium species, and it is likely that these provide T cell help for protective antibody responses against MSP-1₁₉^{11,13}. CD4⁺ T cell lines that recognise epitopes within MSP-1₃₃ (but not MSP-1₁₀ are also capable of controlling growth of lethal P. yoelii following adoptive transfer into immunodeficient mice^{14,15}. Cellular responses to MSP-1 have been found to correlate with protection in studies of semi-immune children³, and similarly, repeated exposure of mice or human volunteers to ultra-low doses of blood-stage parasites induces protective immunity, characterised by cellular rather than antibody responses^{16,17}. CD8⁺ T cells do not contribute to blood-stage immunity in the P. yoelii model¹⁸. However, a T cell response against P. yoelii MSP-1 can protect against the liver-stage parasite¹⁹, since MSP-1 is expressed within the late exo-erythrocytic schizonts²⁰. MSP-1 is thus a target of multi-stage vaccine-induced immunity.

Blood-stage malaria vaccine development has classically focused on recombinant protein-in-adjuvant formulations. These require multiple immunizations in animal models to induce antibody responses of a protective magnitude, and clinical trials of such candidate vaccines remain disappointing⁴. Recombinant protein vaccines are hampered by the inherent difficulties of i) reliably purifying large amounts of correctly folded protein, and ii) formulation in effective human-compatible adjuvants. Viral vaccine vectors, deployed in heterologous prime-boost regimes, have been developed to induce CD8⁺ and CD4⁺ T cell responses targeting intracellular pathogens²¹. They circumvent the difficulties outlined above, and possess greater capacity for the expression of large antigen constructs. Replication-defective poxviruses, such as MVA²², or more recently adenoviruses, such as AdHu5²³, have displayed a suitable safety profile for use in humans as prophylactic vaccines, and have shown excellent efficacy against pre-erythrocytic malaria in mouse models^{24,25}. The induction of strong cellular immune responses, in conjunction with the antibody responses, is one strategy that may enhance blood-stage malaria vaccine efficacy.

Complement protein C4b-binding protein (C4bp), is a circulating soluble inhibitor of the complement pathways²⁶. WO2005/014654 describes the core domain of murine C4bp α-chain (mC4bp) as a molecular adjuvant that may enhance antibody responses when fused to a pathogen antigen. This document discloses that recombinant P. yoelii MSP-1₁₉ fused to mC4bp was highly immunogenic when administered in saline, and induced antibodies of a protective magnitude in mice at a much higher level than when the antigen was injected with Freund's adjuvant.

### SUMMARY OF THE INVENTION

The present invention is based on the novel and unexpected finding that T cell responses to immunogenic compositions and vaccines comprising viral vectors can be enhanced by inclusion of a nucleic acid sequence encoding a C4bp domain in the immunogenic vector encoding the antigen of interest.

The prior art describes fusions of a murine C4bp core domain with monomeric antigens and indicates that these provoke a strong antibody response.

The significant increase in both the CD4 and CD8 T cell responses to immunogenic viral compositions and vaccines seen in the current invention could not have been anticipated by the prior work on antibody responses using C4bp fusion proteins. In fact there are large numbers of adjuvants which can enhance antibody responses but fail to increase CD8 T cell responses, for example, Alum, Montanide, MF59.

Therefore, according to a first aspect of the present invention there is provided an immunogenic composition comprising a viral vector, said vector comprising a nucleic acid sequence encoding a C4bp domain or variant or fragment thereof and a nucleic acid encoding the antigen of interest.

Preferably, the nucleic acid is DNA or RNA.

Preferably, the nucleic acid encoding the C4bp domain is in frame with the nucleic acid encoding the antigen of interest.

It will be apparent that the C4bp domain may be derived from a mammalian or non-mammalian C4bp protein or a fragment thereof.

It will be further apparent that the invention may comprise the use of nucleic acids encoding derivatives of the C4bp core. Such derivatives comprise mutants thereof, which may contain amino acid deletions, additions especially the addition of cysteine residues or substitutions, hybrids or chimeric molecules formed by fusion of parts of different members of the C4bp families and/or circular permutated protein scaffolds, subject to the maintenance of the T cell enhancing properties described herein.

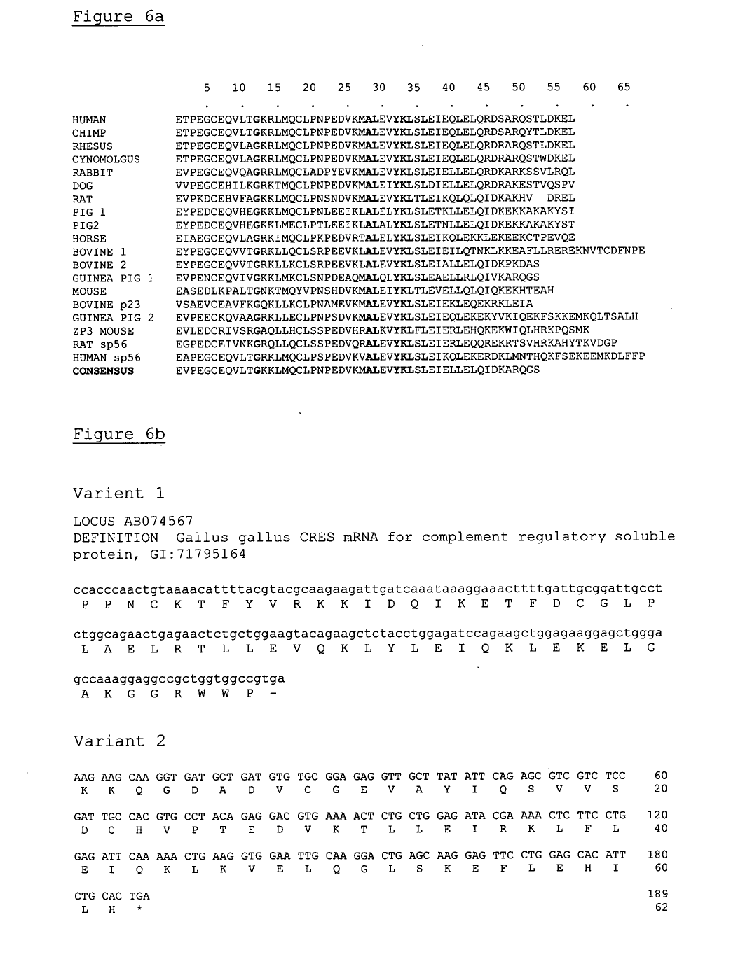

The invention may also use artificial consensus C4bp sequences based on the alignment of the C4bp core sequences from multiple species. One example of this class of chimeric molecule, of the very many possible, is given below in FIG. 6a.

In a particularly preferred embodiment, the nucleic acid encoding C4bp component of the product of the invention encodes the core protein of C4bp alpha chain. In such an embodiment, the nucleic acid encoding the C4bp core encodes a peptide consisting of human C4bp as shown in FIG. 6a or the corresponding residues of a homologue thereof, or a nucleic acid encoding a fragment of at least 47 amino acids of human C4bp or a homologue thereof.

The peptide sequences of a number of mammalian C4bp proteins are available in the art. These include human C4bp core protein. There are a number of homologues of human C4bp core protein also available in the art. There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene, chromosome or genome duplication, i.e. the common ancestor of the genes occurred since the last speciation event.

For example, a search of GenBank and raw genomic trace and EST (expressed sequence tag) databases indicates mammalian C4bp core homologue proteins in species including chimpanzees, rhesus monkeys, rabbits, rats, dogs, horses, mice, guinea pigs, pigs and cattle. Paralogues and orthologues of human C4bp have been included in the alignment in FIG. 6a.

It can be seen that all nineteen sequences have a high degree of similarity, though with a greater degree of variation at the C-terminal end. Further C4bp core proteins may be identified by searching databases of DNA or protein sequences, using commonly available search programs such as BLAST.

Where a nucleic acid encoding C4bp protein from a desired mammalian source is not available in a database, it may be obtained using routine cloning methodology well established in the art. In essence, such techniques comprise using nucleic acid encoding one of the available C4bp core proteins as a probe to recover and to determine the sequence of the C4bp domain from other species of interest. A wide variety of techniques are available for this, for example PCR amplification and cloning of the gene using a suitable source of genomic DNA or mRNA (e.g. from an embryo or an actively dividing differentiated or tumour cell), or by methods comprising obtaining a cDNA library from the mammal, e.g. a cDNA library from one of the above-mentioned sources, probing said library with a known C4bp nucleic acid under conditions of medium to high stringency (for example 0.03M sodium chloride and 0.03M sodium citrate at from about 50° C. to about 60° C.), and recovering a cDNA encoding all or part of the C4bp protein of that mammal. Where a partial cDNA is obtained, the full length coding sequence may be determined by primer extension techniques.

Nucleic acids encoding variants of the C4bp core and fragments thereof may also be used. The variant will preferably have at least 70%, more preferably at least 80%, even more preferably at least 90%, for example at least 95% or most preferably at least 98% sequence identity to the amino acid sequence of a wild type mammalian C4bp core or fragment thereof.

In one aspect, the C4bp core will be a core which includes the glycine appears at position 12, the alanine which appears at position 28, the leucines which appear at positions 29, 34, 36, and 41 and the tyrosine which appears at position 32 and the lysine which appears at position 33 and preferably the two cysteine residues which appear at positions 6 and 18 of human C4bp as shown in FIG. 6a. Desirably, the variant will retain the relative spacing between these residues.

The above-specified degree of identity will be to any one of the sequences shown in FIG. 6a.

The degree of sequence identity may be determined by the algorithm GAP, part of the “Wisconsin package” of algorithms widely used in the art and available from Accelrys (formerly Genetics Computer Group, Madison, Wis.). GAP uses the Needleman and Wunsch algorithm to align two complete sequences in a way that maximises the number of matches and minimises the number of gaps. GAP is useful for alignment of short closely related sequences of similar length, and thus is suitable for determining if a sequence meets the identity levels mentioned above. GAP may be used with default parameters.

Nucleic acid encoding synthetic variants of a mammalian C4bp core protein include those with one or more amino acid substitutions, deletions or insertions or additions to the C- or N-termini. Substitutions are particularly envisaged. Substitutions include conservative substitutions. Examples of conservative substitutions include those respecting the groups of similar amino acids often called the Dayhoff groups. These are as follows:

| | |
|---|---|
| Group 1 | D, E, N, Q |
| Group 2 | I, L, V, M |
| Group 3 | F, Y, W |
| Group 4 | K, R, H |
| Group 5 | S, P, T, A, G |
| Group 6 | C |

Where deletions of the sequence are made, apart from N- or C-terminal truncations, these will preferably be limited to sequences encoding no more than one, two or three deletions which may be contiguous or non-contiguous.

Where insertions are made, or N- or C-terminal extensions to the core protein sequence, these will also be desirably limited in number so that the size of the core protein does not exceed the length of the wild type sequence by more than 20, preferably by no more than 15, more preferably by no more than 10, amino acids. Thus in the case of human C4bp, the nucleic acid encoding the core protein, when modified by insertion or elongation, will desirably encode a peptide of no more than 77 amino acids in length.

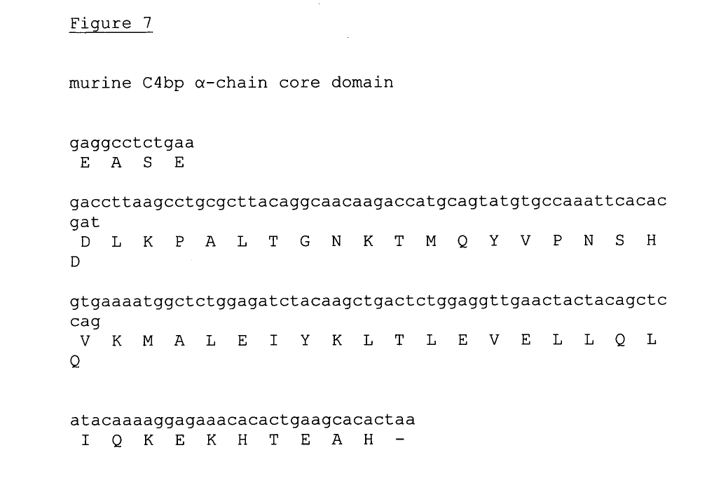

In a further preferred embodiment, the C4bp domain is the murine domain encoded by a sequence as shown in FIG. 7

It will be apparent that for different species, a different c4 bp sequence might be appropriate. It will be understood that the current invention includes viral vectors comprising DNA encoding any of the C4bp domains shown in FIG. 6 or any other C4bp domain.

The composition may be a vaccine composition. Preferably, the vaccine composition is suitable for human administration and can be used to elicit a protective immune response against the encoded antigen.

In a particularly preferred embodiment, when the vaccines are for use in humans, the nucleic acid sequence encodes the variant C4bp domains shown in FIG. 6b. These variants of C4bp overcome the problems of using native C4bp domains, namely that use of a C4bp identical to a fragment of a naturally occurring plasma protein may result not only in a reaction to the C4bp domain in the recombinant protein encoded by the viral vectors, but might also induce a reaction to the endogenous plasma C4bp protein which would not be desirable.

Varient 1 is shown in FIG. 6b is derived from the work of Oshiumi et al. (2005 J. Immunol. 175, 1724-1734). They characterised the regulator of complement activation locus in chicken and identified three proteins which they call CREM, CREG and CRES. Transcripts from each gene were characterised enabling the entire protein sequences to be deduced. One of these proteins, CRES, was described as the chicken C4bp gene and Variant 1 DNA sequence was derived from a cDNA encoding this.

Preferably, the encoded C4bp domain and antigen of interest are expressed as a fusion protein.

Preferably, the viral vector is selected from the group consisting of a poxvirus vector such as modified vaccinia virus Ankara (MVA), an avipox vector such as a fowlpox, canarypox or ALVAC, an herpesvirus vector (including herpes simplex and CMV), an alphavirus vector and an adenovirus vector. More preferably, the viral vector is a pox virus vector or an adenovirus vector. Even more preferably, the vector is MVA or AdHu5.

It will be apparent that the antigen can be any antigen of interest either exogenous or endogenous. Exogenous antigens include all molecules found in infectious organisms. For example bacterial immunogens, parasitic immunogens and viral immunogens.

Bacterial sources of these immunogens include those responsible for bacterial pneumonia, meningitis, cholera, diphtheria, pertussis, tetanus, tuberculosis and leprosy.

Parasitic sources include malarial parasites, such as Plasmodium, as well as trypanosomal and leishmania species.

Viral sources include poxviruses, e.g., smallpox virus, cowpox virus and orf virus; herpes viruses, e.g., herpes simplex virus type 1 and 2, B-virus, varicella zoster virus, cytomegalovirus, and Epstein-Barr virus; adenoviruses, e.g., mastadenovirus; papovaviruses, e.g., papillomaviruses such as HPV16, and polyomaviruses such as BK and JC virus; parvoviruses, e.g., adeno-associated virus; reoviruses, e.g., reoviruses 1, 2 and 3; orbiviruses, e.g., Colorado tick fever; rotaviruses, e.g., human rotaviruses; alphaviruses, e.g., Eastern encephalitis virus and Venezuelan encephalitis virus; rubiviruses, e.g., rubella; flaviviruses, e.g., yellow fever virus, Dengue fever viruses, Japanese encephalitis virus, Tick-borne encephalitis virus and hepatitis C virus; coronaviruses, e.g., human coronaviruses; paramyxoviruses, e.g., parainfluenza 1, 2, 3 and 4 and mumps; morbilliviruses, e.g., measles virus; pneumovirus, e.g., respiratory syncytial virus; vesiculoviruses, e.g., vesicular stomatitis virus; lyssaviruses, e.g., rabies virus; orthomyxoviruses, e.g., influenza A and B; bunyaviruses e.g., LaCrosse virus; phleboviruses, e.g., Rift Valley fever virus; nairoviruses, e.g., Congo hemorrhagic fever virus; hepadnaviridae, e.g., hepatitis B; arenaviruses, e.g., lcm virus, Lasso virus and Junin virus; retroviruses, e.g., HTLV I, HTLV II, HIV-1 and HIV-2; enteroviruses, e.g., polio virus 1,-2 and 3, coxsackie viruses, echoviruses, human enteroviruses, hepatitis A virus, hepatitis E virus, and Norwalk-virus; rhinoviruses e.g., human rhinovirus; and filoviridae, e.g., Marburg (disease) virus and Ebola virus.

Antigens from these bacterial, viral and parasitic sources can be considered as exogenous antigens because they are not normally present in the host and are not encoded in the host genome.

In contrast, endogenous antigens are normally present in the host or are encoded in the host genome, or both. The ability to generate an immune response to an endogenous antigen is useful in treating tumours that bear that antigen, or in neutralising growth factors for the tumour. An example of the first type of endogenous antigen is HER2, the target for the monoclonal antibody called Herceptin. An example of the second, growth factor, type of endogenous antigen is gonadotrophin releasing hormone (called GnRH) which has a trophic effect on some carcinomas of the prostate gland.

Immunogenic compositions or vaccines formed according to the invention may be used for simultaneous vaccination against more than one disease, or to target simultaneously a plurality of epitopes on a given pathogen.

In one preferred embodiment, the antigen is a malaria antigen. Preferably, the antigen is a P. falciparum antigen. More preferably, the antigen is ME-TRAP, CSP, MSP-1 or fragments thereof, or AMA1. Most preferably, the antigen is a blood-stage malarial antigen.

It will be apparent to the skilled person that the above immunogenic compositions or vaccines, may be formulated into pharmaceutical dosage forms, together with suitable pharmaceutically acceptable vehicles or carriers, such as diluents, fillers, salts, buffers, stabilizers, solubilizers, etc. The dosage form may contain other pharmaceutically acceptable excipients for modifying conditions such as pH, osmolarity, taste, viscosity, sterility, lipophilicity, solubility etc and may also contain pharmaceutically acceptable adjuvants.

Suitable dosage forms include solid dosage forms, for example, tablets, capsules, powders, dispersible granules, cachets and suppositories, including sustained release and delayed release formulations. Powders and tablets will generally comprise from about 5% to about 70% active ingredient. Suitable solid carriers and excipients are generally known in the art and include, e.g. magnesium carbonate, magnesium stearate, talc, sugar, lactose, etc. Tablets, powders, cachets and capsules are all suitable dosage forms for oral administration.

Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Suitable diluents and solvents include sterile water, Ringer's solution and isotonic sodium chloride solution, etc. Liquid dosage forms also include solutions or sprays for intranasal administration.

Aerosol preparations suitable for inhalation may include solutions and solids in powder form, which may be combined with a pharmaceutically acceptable carrier, such as an inert compressed gas.

Also encompassed are dosage forms for transdermal administration, including creams, lotions, aerosols and/or emulsions. These dosage forms may be included in transdermal patches of the matrix or reservoir type, which are generally known in the art.

Pharmaceutical preparations may be conveniently prepared in unit dosage form, according to standard procedures of pharmaceutical formulation. The quantity of active compound per unit dose may be varied according to the nature of the active compound and the intended dosage regime.

The active agents are to be administered to human subjects in “therapeutically effective amounts”, which is taken to mean a dosage sufficient to provide a medically desirable result in the patient. The exact dosage and frequency of administration of a therapeutically effective amount of active agent will vary, depending on such factors as the nature of the active substance, the dosage form and route of administration.

The medicaments and pharmaceutical compositions of the present invention may be administered systemically or locally. This is applicable to both the use and method aspects of the invention equally. Systemic administration may be by any form of systemic administration known, for example, orally, intravenously or intraperitoneally. Local administration may be by any form of local administration known, for example topically.

In particularly preferred embodiments the pharmaceutical composition includes at least one pharmaceutically acceptable excipient.

According to a second aspect of the present invention there is provided a product, combination or kit comprising;

a) a priming composition comprising a first viral vector, said viral vector further comprising a nucleic acid encoding a C4bp domain or variant or fragment thereof, and at least one pathogen or tumour antigen; and
b) a boosting composition comprising a second viral vector, said second viral vector being different from said first viral vector and further comprising a nucleic acid encoding a C4bp domain, and at least one pathogen or tumour antigen which is the same as the pathogen or tumour antigen of the priming composition,

Preferably, the nucleic acid encoding the C4bp domain is in frame with the nucleic acid encoding the antigen of interest.

Preferably, the C4bp domain is one of those shown in FIG. 6.

Preferably, the C4bp domain is encoded by a sequence as shown in FIG. 7

Preferably, the C4bp domain and antigen of interest are expressed as a fusion protein.

Preferably, each viral vector is selected from the group consisting of a poxvirus vector such as modified vaccinia virus Ankara (MVA), an avipox vector such as a fowlpox, canarypox or ALVAC, an herpesvirus vector (including herpes simplex and CMV), an alphavirus vector and an adenovirus vector.

Preferably, said first viral vector is an adenoviral vector. More preferably, AdHu5.

Preferably, said second viral vector is a pox virus vector. More preferably, MVA.

It will be apparent to the skilled person that the antigen of interest can be any suitable antigen, as described in relation to the first aspect.

In one preferred embodiment, the antigen is a malaria antigen. Preferably, the antigen is a P. falciparum antigen. More preferably, the antigen is ME-TRAP, CSP, MSP-1 or fragments thereof, or AMA1. Most preferably, the antigen is a blood-stage malarial antigen.

Also provided, is the use of the product, combination or kit for production of a kit for generating a protective T cell response against at least one target antigen of a pathogen or tumour in a subject.

It will be apparent that the subject can be any animal subject. In a particularly preferred embodiment this may be a mammalian subject, including a human. In an alternate embodiment, the subject may be an avian subject.

According to a third aspect of the current invention there is provided a viral vector comprising a nucleic acid sequence encoding a C4bp domain or variant or fragment thereof and a nucleic acid encoding the antigen of interest.

Preferably, the nucleic acid encoding the C4bp domain is in frame with the nucleic acid encoding the antigen of interest.

Preferably, the C4bp domain is one of those shown in FIG. 6.

Preferably, the C4bp domain is encoded by a sequence as shown in FIG. 7

Preferably, the C4bp domain and antigen of interest are expressed as a fusion protein.

Preferably, the viral vector is selected from the group consisting of a poxvirus vector such as modified vaccinia virus Ankara (MVA), an avipox vector such as a fowlpox, canarypox or ALVAC, an herpesvirus vector (including herpes simplex and CMV), an alphavirus vector and an adenovirus vector. More preferably, the viral vector is a pox virus vector or an adenovirus vector. Even more preferably, the vector is MVA or AdHu5.

It will be apparent to the skilled person that the antigen of interest can be any suitable antigen, as described in relation to the first aspect.

In one preferred embodiment, the antigen is a malaria antigen. Preferably, the antigen is a P. falciparum antigen. More preferably, the antigen is ME-TRAP, CSP, MSP-1 or fragments thereof, or AMA1. Most preferably, the antigen is a blood-stage malarial antigen

According to a fourth aspect of the current invention there is provided a viral vector according to the third aspect for use as a vaccine.

According to a fifth aspect of the present invention there is provided the use of a vector according to the third aspect in the manufacture of a vaccine for the prevention or treatment of malaria.

According to a sixth aspect there is provided a vector according to the third aspect for use in the prevention or treatment of malaria.

According to a seventh aspect of the present invention there is provided a method of immunising a subject by administering an effective amount of at least one immunogenic composition or vaccine according to the first aspect of the present invention.

It will be apparent that the subject can be immunised for either prophylactic or immunotherapeutic purposes, depending on the antigen.

In a preferred embodiment, the subject is immunised using a heterologous prime-boost regimen, wherein there are provided at least two immunogenic composotions or vaccines according to the first aspect wherein the second immunogenic composition or vaccine according to the first aspect is administered subsequently to the first immunogenic composition or vaccine according to the first aspect.

The skilled person will understand that heterologous prime-boost refers to a regimen wherein a first unit dose of the immunogenic composition or vaccine according to the present invention is administered to an individual at a first time point and subsequently a second unit dose of the immunogenic composition or vaccine according to the present invention is administered at a second time point. It will be understood that in a heterologous prime-boost regimen the viral vectors forming the immunogenic composition or vaccine in the first and second unit doses are different.

Preferably, the immunogenic composition or vaccine forming the first unit dose comprises an adenoviral vector. More preferably, AdHu5.

It will be further understood that in many applications according to any of the previous aspects it is preferable for the adenovirus vector to be replication deficient meaning that they have been rendered incapable of replication because of a functional deletion, or complete removal, of a gene encoding a gene product essential for viral replication. By way of example, the vectors of the invention may be rendered replication defective by removal of all or a part of the E1 gene, and optionally also the E3 region and/or the E4 region.

Preferably, the immunogenic composition or vaccine forming the second unit dose comprises a poxyiral vector. More preferably, MVA.

Preferably, the time period between administration of the first and second unit doses is 2-8 weeks.

It will be readily apparent to the skilled person that the term subject as used in the present invention relates to any animal subject. This may particularly be a mammalian subject, including a human.

Thus products of the invention may be useful not only in human use but also in veterinary uses, for example in the treatment of domesticated mammals including livestock (e.g. cattle, sheep, pigs, goats, horses) and pets (e.g. cats, dogs, rodents) or in the treatment of wild mammals, such as those captive in zoos.

In another aspect, the product of the invention may be used for the treatment of non-mammalian subjects, including fowl such as chickens, turkeys, duck, geese and the like.

It will be understood that immunising a subject with an immunogenic composition or vaccine according to any aspect of the present invention either singly, or using a prime boost regime results in an enhanced T cell response to the antigen when compared to immunising a subject with an immunogenic composition or vaccine not including the C4bp domain.

The invention will now be described with reference to the following figures and example in which:—

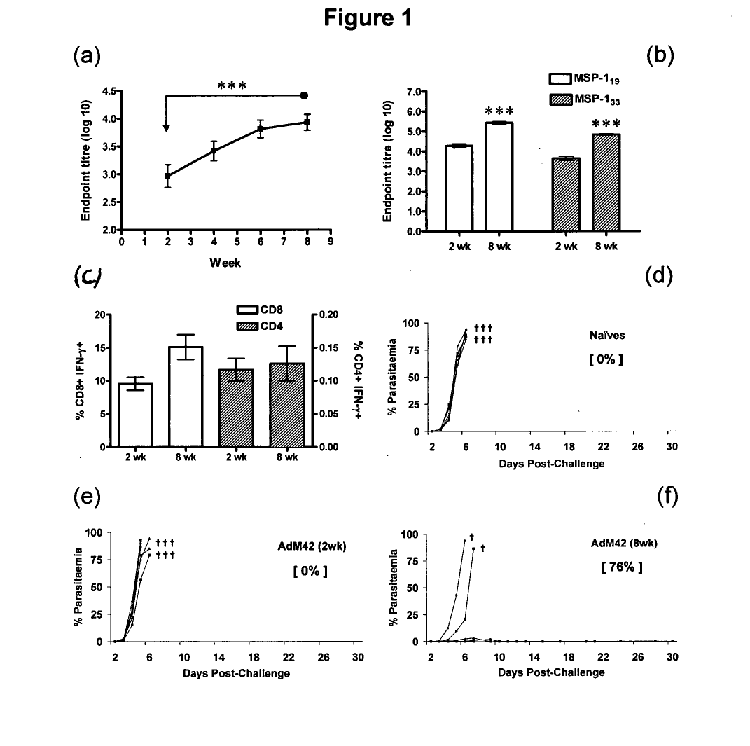

FIG. 1 shows that AdM42 vaccine-induced immune responses and protection against blood-stage P. yoelii are dependent on prime-boost interval. (a) BALB/c mice were immunized with Ad42 and whole IgG serum antibody responses against MSP-1₁₉ were measured over time by ELISA. (b) Mice were primed with Ad42 and boosted with M42 two or eight weeks later. Whole IgG serum antibody responses against MSP-1₁₉ and MSP-1₃₃ were measured by ELISA 14 days post-boost. (c) CD8⁺ and CD4⁺ T cell IFN-γ responses following re-stimulation with MSP-1₃₃ OLP were assessed in the spleen by ICS. The mean responses±SEM are shown for each time point (n≧6 mice per group). *** p≦0.001 comparing responses between time-points in (a), or between the two groups in (b). Similar results were obtained in two or three independent experiments. (d) Naïve unimmunized control mice, (e) AdM42 (2 wk) or (f) AdM42 (8 wk) immunized mice were challenged with 10⁴ P. yoelii pRBCs on day 14 post-boost. Parasitemia was measured as described from day two post-challenge. Representative results are shown (n=6 mice per group). Percentage survival, including all repeat experiments, is indicated in square parentheses see Table 1a.

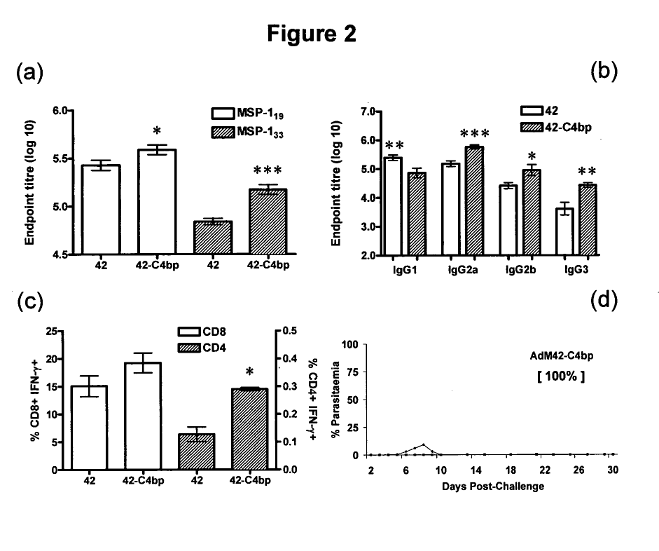

FIG. 2 shows AdM42-C4bp vaccine-induced immune responses and protection against blood-stage P. yoelii. BALB/c mice were immunized with either AdM42 or AdM42-C4bp regimes, using an eight week prime-boost interval. (a) Whole IgG serum antibody responses against MSP-1₁₉ and MSP-1₃₃, or (b) IgG isotype responses against MSP-1₁₉ᵣ were measured by ELISA 14 days post-boost. (c) CD8⁺ and CD4⁺ T cell IFN-γ responses were assessed in the spleen as before. The mean responses±SEM are shown for each time point assayed (n≧6 mice per group). * p≦0.05, ** p≦0.01, *** p≦0.001 comparing responses between the two groups. Similar results were obtained in two or three independent experiments. (d) AdM42-C4bp immunized mice were challenged as before with 10⁴ P. yoelii pRBCs. A representative result is shown (n=6 mice per group). Percentage survival, including all repeat experiments, is indicated in square parentheses (see Table 1b).

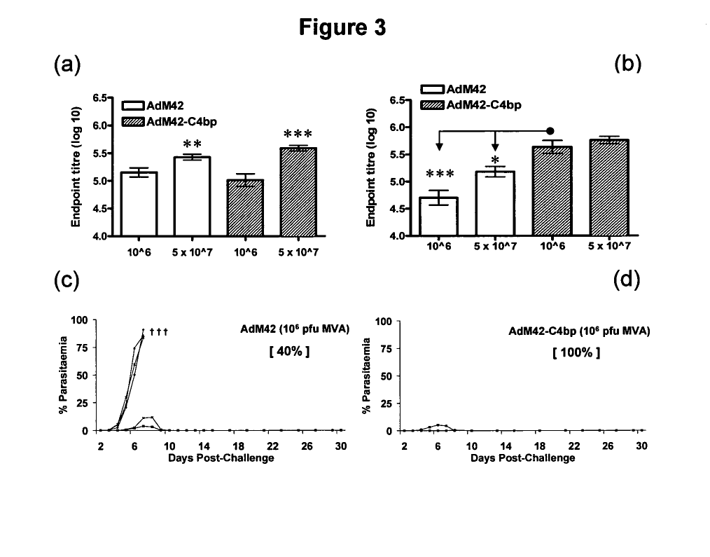

FIG. 3 shows that protection against P. yoelii pRBC challenge correlates with the level of vaccine-induced MSP-1₁₉-specific IgG2a. BALB/c mice were immunized with the AdM42 or AdM42-C4bp regimes, using either 5×10⁷ pfu or 10⁶ pfu MVA to boost. (a) Whole IgG serum antibody responses against MSP-1₁₉ or (b) IgG2a responses against MSP-1₁₉, were measured by ELISA 14 days post-boost. The mean responses±SEM are shown (n≧6 mice per group). * p≦0.05, ** p≦0.01, *** p≦0.001 comparing responses between the two groups receiving the same vaccine in (a), or comparing between all groups in (b). Similar results were obtained in two independent experiments. Mice immunized with (c) AdM42 or (d) AdM42-C4bp, using 10⁶ pfu MVA to boost, were challenged as before with 10⁴ P. yoelii pRBCs. Representative results are shown (n=5 or 6 mice per group). Percentage survival is indicated in square parentheses (see Table 1d).

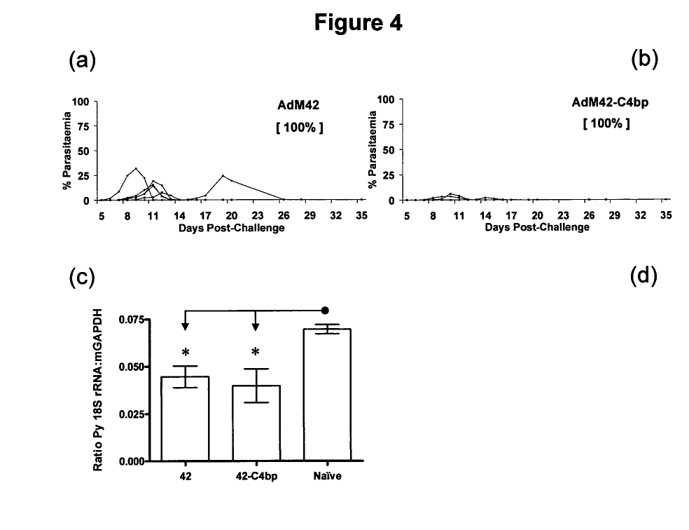

FIG. 4 shows that AdHu5-MVA immunization can protect against P. yoelii sporozoite challenge and against the liver-stage exo-erythrocytic forms. BALB/c mice were immunized with (a) AdM42 or (b) AdM42-C4bp, using 5×10⁷ pfu MVA to boost, and were challenged with 50 P. yoelii sporozoites on day 14 post-boost (n=6 mice per group). Percentage survival is indicated in square parentheses (see Table 1e). (c) BALB/c mice were immunized with AdM42 or AdM42-C4bp and challenged with 5,000 P. yoelii sporozoites. 48 hours later livers were harvested and total RNA extracted and converted to cDNA. Liver-stage parasite burden was assessed by real-time RT-PCR and is expressed as the ratio of the number of copies P. yoelii 18S rRNA normalised to the number of copies of murine GAPDH.

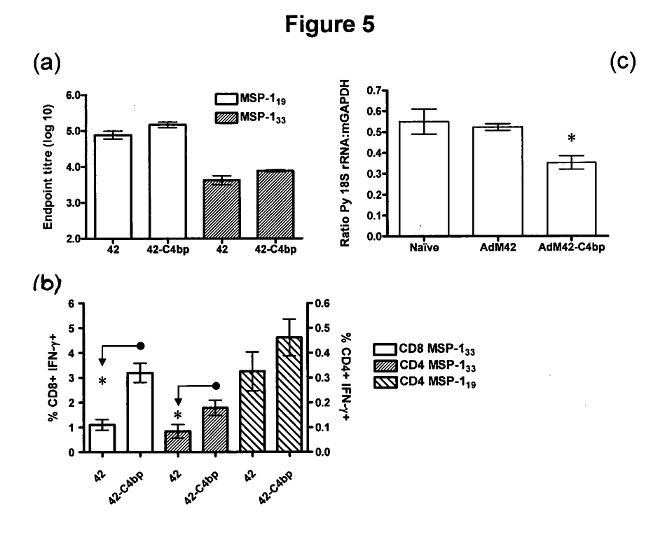

FIG. 5 shows Immunogenicity and protective efficacy of AdM42 and AdM42-C4bp immunization in C57BL/6 mice. Mice were immunized with either the AdM42 or AdM42-C4bp regimes. Immune responses were assessed 14 days post-boost. (a) Whole IgG serum antibody responses against MSP-1₁₉ and MSP-1₃₃. (b) CD8⁺ and CD4⁺ T cell IFN-γ responses following re-stimulation with MSP-1₃₃ or MSP-1₁₉ OLP were assessed in the spleen by ICS. The mean responses±SEM are shown for each response (n≧6 mice per group). *** p≦0.001 comparing responses between the two groups. (c) Mice were immunized and challenged with 5,000 P. yoelii sporozoites. 48 hours later liver-stage parasite burden was assessed as before. The mean ratio±SEM is shown (n=5 mice per group). * p≦0.05 comparing between all groups.

FIG. 6a shows an alignment of the amino acid sequences of C4bp domains from various mammalian species.

FIG. 6b shows the DNA and deduced amino acid sequences of two variant C4bp domains.

FIG. 7 shows the DNA and deduced amino acid sequence of the murine C4bp domain.

### EXAMPLES

### Materials and Methods

Generation of Recombinant MVA and AdHu5 Vaccines Expressing MSP-1₄₂. P. yoelii YM MSP-1₄₂ (amino acids (aa) 1394-1757) was amplified by Expand High Fidelity PCR (Roche) using the 42F forward primer 5′-GTC GAC TCC GAA GAT GCA CCA GAA AAA GAT AT-3′ and the 42R reverse primer 5′-GCA TGC GGA TCC TCA GTC TAG ACC TAG CAA AGG GTT AGG AAT TCC CAT AAA GCT GGA AGA ACT ACA GAA TAC-3′ from plasmid XPyM4.3²⁷ a gift from Dr A. A. Holder (NIMR, U.K.). The primers included a 5′ Sal 1 and 3′ BamH I and Sph I restriction sites, and TGA stop codon. 42R also encodes a C-terminal anti-PK monoclonal antibody recognition sequence IPNPLLGLD. The C-terminal GPI anchor signal sequence (aa 1758-1773) of MSP-1 was excluded. The PCR product was ligated into pGEM-T Easy (Promega) and verified by sequence analysis (MWG Biotech, Ebersberg, Germany). The human tissue plasminogen activator (tPA) leader sequence was amplified by PCR from a plasmid template²⁸ using the forward primer 5′-GGA TCC GCG CGC CGC CAC C-3′ and the reverse primer 5′-CTC GAG TCT TCT GAA TCG GGC ATG G-3′. The primers included 5′ BamH I and 3′ Xho I restriction sites, as well as the Kozak sequence (underlined). The product was cloned and sequenced as above. The Sal I-MSP-1₄₂—PK-Sph I construct was cloned into the tPA vector to create an in-frame fusion with the N-terminal leader sequence.

The entire construct, now contained between two BamH I sites, was cloned into the Bgl II site of the MVA shuttle vector, pMVA.GFP. This vector, pMVA.GFP.MSP-1₄₂, drives antigen expression using the vaccinia P7.5 early/late promoter, and expression of GFP from the fowlpox late promoter, FP4b. Primary chicken embryo fibroblasts (CEFs, SPF grade) were infected with MVA.Red, which expresses Red Fluorescent Protein (RFP) driven by the Vaccinia P7.5 promoter, integrated at the TK locus of MVA. The infected cells were then transfected with 2 μg Hind III linearised pMVA.GFP.MSP-1₄₂ using Lipofectin (Invitrogen, Paisley, U.K.). CEFs infected with recombinants expressing GFP (in which MSP-1₄₂ and GFP had replaced RFP at the TK locus following homologous recombination) were enriched from those infected with MVA-RFP using a fluorescence-activated cell sorter. Pure recombinant virus was isolated by repeated plaque picking in CEFs using the GFP marker as visualised by fluorescence microscopy.

Recombinant AdHu5 vaccines were constructed using the ViraPower Adenoviral expression system (Invitrogen). The 1.9 kbp CMV promoter (with regulatory element, enhancer and intron A), polylinker and BGH poly(A) transcription termination sequence from the DNA vaccine vector pSG2²⁹ was cloned into the entry vector pENTR4 (Invitrogen). The MSP-1₄₂ construct was cloned into the BamH I site of pENTR4.CMV-BGH. This construct was recombined using LR clonase enzyme mix (Invitrogen) into the 36 kbp E1- and E3-deleted pAd/PL-DEST AdHu5 genome vector (Invitrogen). Vectors were linearised with Pac I to expose the inverted terminal repeats and transfected into 293A cells (Invitrogen). Pure recombinant AdHu5 viruses were grown out as per manufacturer's instructions. Recombinant adenovirus was purified using the Adenopure Kit (PureSyn, Malvern, Pa., USA). Pure virus was aliquoted and stored at −80° C. Absorbance at 260 nm of virus stock (diluted 1:100 in water) was measured using a UV spectrophotometer, and the viral particle (vp) count was calculated using the formula: Abs₂₆₀×10¹²× dilution factor=vp/mL

### Generation of Recombinant MVA and AdHu5 Vaccines Expressing MSP-1₄₂—C4bp.

The core domain of the murine C4bp protein alpha chain (aa 416-469) and a TAA stop codon were cloned into the pMVA.GFP.MSP-1₄₂ vector by PCR. The construct was sequenced to confirm an in-frame fusion. The final three amino acids of the original MSP-1₄₂ construct (amino acids 1755-1757) were excluded, as was the C-terminal PK epitope sequence. Recombinant MVA and AdHu5 were generated as described.

### Animals and Immunizations.

Female BALB/c (H-2^{d}) and C57BL/6 (H-2^{b}) mice (BMSU, John Radcliffe Hospital, Oxford, U.K.), 5-6 weeks old, were used in all experiments. All procedures were carried out under the terms of the U.K. Animals (Scientific Procedures) Act Home Office Project Licence. Mice were immunized intradermally (i.d.) with 10⁶ pfu or 5×10⁷ pfu MVA vaccines, or 5×10¹⁰ vp AdHu5 vaccines, diluted in endotoxin-free PBS and administered bilaterally into the ears.

### Generation of Recombinant GST-Fusion Proteins.

P. yoelii YM MSP-1₁₉ (aa 1649-1757) was amplified by PCR as above using the 19F forward primer 5′-GGA TCC GTC GAC ATG GAT GGT ATG GAT TTA TTA GGT G-3′ and the 42R reverse primer. MSP-1₁₉ sequence was excised from the pGEM-T Easy-MSP-1₁₉—PK vector by BamH I and EcoR I restriction enzyme digest. EcoR I conveniently cuts at the end of the MSP-1₁₉ coding sequence, exactly prior to the PK tag. This fragment was cloned into the glutathione S-transferase (GST)-fusion protein expression vector pGEX-2T (Amersham Biosciences, Bucks., U.K.), before transformation into Rosetta Escherichia coli cells (Novagen, Nottingham, U.K.). The proteins were produced as described previously¹⁰ with some modifications. Briefly, overnight cultures of transformed bacteria were induced with 0.25 mM isopropyl-β-D-thiogalactopyranoside (IPTG). Cells were harvested and lysed using BugBuster and benzonase endonuclease (Novagen), and then ultracentrifuged. Recombinant protein was purified by affinity chromatography from the clarified extract using the GST.Bind Purification Kit (Novagen) as per manufacturer's instructions. Recombinant GST-MSP-1₃₃ and GST control¹⁰ were a kind gift from Dr A. A. Holder and Dr I. T. Ling (NIMR, U.K.).

### ELISA.

Serum was collected from tail vein blood samples as previously described³⁰. Recombinant GST fusion protein or GST control were adsorbed overnight at room temperature (RT) to 96 well Nunc-Immuno Maxisorp plates (Fisher Scientific) at a concentration of 2 μg/mL in PBS. Serum was analysed for antibodies by indirect ELISA as previously described³⁰ with some modifications. Briefly, plates were washed with PBS containing 0.05% Tween 20 (PBS/T) and blocked with 10% skimmed milk powder in PBS/T for 1 h at RT. Sera were typically diluted to 1:100, added in duplicate wells and serially diluted. Following 2 h incubation at RT, bound antibodies were detected using alkaline phosphatase-conjugated goat anti-mouse whole IgG (Sigma), or biotin-conjugated rat anti-mouse IgG1, IgG2a, IgG2b or IgG3 (BD Biosciences) followed by incubation with ExtrAvidin alkaline phosphatase conjugate (Sigma) for isotype analysis. Plates were developed by adding p-nitrophenylphosphate substrate (Sigma). Optical density was read at 405 nm (OD₄₀₅). Endpoint titres were taken as the x-axis intercept of the dilution curve at an absorbance value 3× standard deviations greater than the OD₄₀₅ for naïve mouse serum (typical cut-off OD₄₀₅ for positive sera=0.15). A high-titre serum sample was included in all assays as a reference control.

### Intracellular Cytokine Staining (ICS).

Specific IFN-γ secretion by mouse splenocytes was assayed by ICS, as described previously²⁵. Briefly, splenocytes were re-stimulated in the presence of GolgiPlug (BD Biosciences) for 5 h at 37° C. with pools of 15-mer peptides overlapping by 10 aa (final concentration 5 μg/mL each peptide). Overlapping peptide (OLP) pools corresponded to MSP-1₃₃ (aa 1394-1663) containing 52 peptides, and MSP-1₁₉ (aa 1654-1757) containing 19 peptides. Cells were resuspended in PBS and Fc receptors blocked using 5 μg/mL anti-CD16/CD32 (Ebiosciences). Cells were washed twice in PBS and surface stained for 30 min at 4° C. with peridinin chlorophyll-a protein-cyanin 5.5 (PerCP-Cy5.5)-labelled anti-CD8α (clone 53-6.7, BD Biosciences) and fluorescein isothiocyanate (FITC)-labelled anti-CD4 (clone GK1.5, Ebiosciences). Cells were washed twice with PBS, and then permeabilised for 20 min at 4° C. in 100 μL Cytofix/Cytoperm solution (BD Biosciences). Cells were washed twice with Perm/Wash solution and then stained for 30 min at 4° C. with allophycocyanin (APC)-conjugated anti-mouse IFN-γ (clone XMG1.2, Ebiosciences). Cells were washed twice in Perm/Wash solution and resuspended in 150 μL PBS containing 1% formalin. Samples were analysed using a FACSCanto (BD Biosciences, Oxford, UK) and FlowJo software. Background responses in unstimulated control cells were all ≦0.05%, and these values were subtracted from the stimulated response.

### In Vivo Depletions.

In vivo depleting monoclonal antibodies (mAbs), were purified by protein G affinity chromatography from hybridoma culture supernatants. Anti-CD4 GK1.5 (rat IgG2a) and anti-CD8 2.43 (rat IgG2a) were diluted in sterile PBS. Normal rat IgG (nRatIgG) was purchased from Sigma and purified by the same method. For depletion of CD4⁺ or CD8⁺ T cells, mice were injected intraperitoneally (i.p.) with 200 μg of the relevant mAb on days −2 and −1 before, and on the day of challenge. Mice were further administered the same dose of mAbs on days +7, +14 and +21 post-challenge. The degree of in vivo T cell depletion was assessed by flow cytometry of surface-stained splenocytes, from depleted and control mice. Cells were surface-stained, as above, using PerCP-Cy5.5-conjugated anti-mouse CD8α (clone 53-6.7), FITC-labelled anti-mouse CD4 (clone RM4-5), and APC-conjugated anti-mouse CD3ε (clone 145-2C11, Ebiosciences).

P. yoelii pRBC and Sporozoite Challenge.

P. yoelii parasites (strain YM) were kindly provided by Dr G. A. Butcher (Imperial College, London, U.K.) and were kept frozen or regularly passaged in mice. For blood-stage challenge, mice were infected with 10⁴ parasitized red blood cells (pRBCs) by the intravenous (i.v.) route. Parasitemia was monitored from day two post-challenge by microscopic examination of Giemsa-stained blood smears. Levels of parasitemia were assessed by light microscopy and calculated as the percentage of pRBCs. Mice were deemed uninfected in the absence of patent parasitemia in 50 fields of view. For sporozoite challenge, salivary glands of infected female Anopheles stephensi mosquitoes were dissected and homogenised in RPMI 1640 medium to release parasites. Mice were challenged with 50 sporozoites by the i.v. route, and blood-stage parasitemia was monitored from day 5 as above.

Quantification of P. yoelii Parasite Burden in the Liver.

Mice were challenged as above with 5,000 sporozoites. Livers were harvested after 48 h and snap frozen in liquid nitrogen. Whole livers were homogenised in 8 mL Trizol (Invitrogen). Total liver RNA was extracted using chloroform as previously described³¹ and quantified using a nanodrop. 40 μg RNA was digested with RNase-free DNase (Qiagen) and purified using RNeasy MinElute Cleanup Kit (Qiagen). 2 μg RNA was reverse transcribed to cDNA using Omniscript (Qiagen), random hexamer primers (Promega), oligo-dT, and RNasin Plus inhibitor (Promega). cDNA encoding P. yoelii 18S rRNA or mouse glyceraldehyde-3-phosphate dehydrogenase (mGAPDH) were amplified in triplicate by quantitative real-time PCR using a Rotor-Gene 3000 (Corbett Life Science, Sydney, Australia). 20 pmoles of either specific primer pair (PyF 5′-GGG GAT TGG TTT TGA CGT TTT TGC G-3′ and PyR 5′-AAG CAT TAA ATA AAG CGA ATA CAT CCT TAT-3′; mGF 5′-TTC ACC ACC ATG GAG AAG GC-3′ and mGR 5′-GGC ATG GAC TGT GGT CAT GA-3′) were included in the QuantiTect RT-PCR buffer (Qiagen) containing the dsDNA-specific fluorescent dye SYBR Green I. The temperature profile of the reaction was previously described³². The threshold cycle value (C_{T}) of each PCR was converted to a DNA copy number equivalent by reading against standard curves generated by amplifying 10-fold dilutions of pGEM-T Easy plasmid containing the relevant target cDNA molecule. The liver-stage parasite burden was determined for each sample as the ratio of the DNA copy number equivalent measured for the P. yoelii 18S rRNA over the DNA equivalent for mGAPDH.

### Statistical Analysis.

Data were analyzed using GraphPad Prism for windows v4

(GraphPad Software Inc.). Independent-samples or paired Student's t-test were performed, as appropriate, to compare mean responses between two groups. One-way between-groups ANOVA with post-hoc Bonferroni or Dunnett's correction, as appropriate, was used to compare responses between more than two groups. A value of p≦0.05 was considered to be significant in all cases.

### Results

Vaccine-Induced Antibody Responses and Protection Against Blood-Stage P. yoelii are Enhanced when Using an Extended AdHu5-MVA Prime-Boost Interval.

T cell induction by heterologous prime-boost immunization using replication-defective viruses remains a leading vaccine strategy in the fields of HIV²³, malaria^{21,24,25} and tuberculosis³³. AdHu5 and MVA vaccines can also induce antigen-specific antibodies^{34,35}. One study using AdHu5 prime and vaccinia virus boost reported enhanced antibody responses to a sporozoite antigen when the prime-boost interval was extended from two to eight or more weeks³⁶.

We prepared AdHu5 and MVA vaccines expressing MSP-1₄₂ (Ad42 and M42 respectively) from the murine malaria P. yoelii. BALB/c mice were immunized once i.d. with 5×10¹⁰ vp Ad42, and MSP-1₁₉-specific antibody responses were monitored by ELISA over the following 56 days. As shown in FIG. 1a Whole IgG antibody responses against MSP-1₁₉ were significantly higher at day 56 compared to day 14. To assess immune responses following AdHu5-MVA prime-boost (AdM42), mice were primed with Ad42 and then boosted i.d. with 5×10⁷ pfu M42 at day 14 (2 wk) or day 56 (8 wk). Cellular and humoral immune responses against MSP-1₄₂ were examined 14 days after the boost. FIG. 1b shows that significantly higher whole IgG responses against MSP-1₁₉ and MSP-1₃₃ were induced following the eight week prime-boost regime. The antibody responses achieved against MSP-1₁₉ in the case of the AdM42 (8 wk) regime were very strong exhibiting endpoint titres over 10⁵. No intracellular IFN-γ production was detected in splenic T cells following re-stimulation with the pool of OLP corresponding to MSP-1₁₉ (data not shown). FIG. 1c shows that strong CD8⁺ IFN-γ⁺ T cell responses were measured against MSP-1₃₃. These tended to increase in the AdM42 (8 wk) group but were not significantly higher when compared to the two week regime. FIG. 1c also shows that relatively weak CD44⁺ IFN-γ⁺ T cell responses could be measured against MSP-1₃₃ in both groups.

To determine the protective efficacy of AdM42 vaccination, mice were challenged intravenously with 10⁴ pRBCs two weeks post-boost as shown in Table 1a at the end of specification. FIGS. 1d and 1e show that all of the naïve unimmunized control mice and those that received the AdM42 (2 wk) prime-boost regime succumbed to P. yoelii infection within six days. FIG. 1f shows that the AdM42 (8 wk) regime protected 76% mice against challenge. Vaccine-mediated blood-stage protection correlated with the significantly higher levels of MSP-1₄₂-specific IgG achieved by using an extended prime-boost interval.

AdM42-C4bp Immunization Significantly Enhances Th1-type Antibody and CD4 T Cell Responses, and Improves Protective Efficacy Against P. yoelii pRBC Challenge.

The use of mC4bp and another complement component-based “molecular adjuvant”, C3d, has been shown in WO2005/014654 to be effective when using recombinant fusion protein vaccines³⁷, and also plasmid DNA vaccines³⁸. However, prior to the present invention, the expression of such constructs by viral vaccine vectors has not been described.

The inventors produced AdHu5 and MVA vectors expressing MSP-1₄₂ fused at the C-terminus to mC4bp. BALB/c mice were immunized using an eight week prime-boost interval (AdM42 versus AdM42-C4bp) and immune responses assayed as before. FIG. 2a shows that mice immunized with vectors expressing MSP-1₄₂—C4bp developed significantly higher antigen-specific whole IgG responses. The inventors focussed on the isotype profile of the IgG induced against the protective MSP-1₁₉ domain¹⁰. Remarkably, as shown in FIG. 2b the use of vectors expressing MSP-1₄₂—C4bp led to a Th1-shift in antigen-specific IgG, with significantly elevated levels of IgG2a, IgG2b and IgG3, and reduced levels of IgG1, when compared to mice immunized with vectors expressing MSP-1₄₂. No differences in antibody avidity³⁸ were observed between the two groups (data not shown). FIG. 2c shows that there was also a significant three-fold increase in the percentage of MSP-1₃₃-specific splenic CD4⁺ IFN-γ⁺ T cells in the mice immunized with the MSP-1₄₂-C4bp vectors; possibly accounting for the enhanced levels of Th1-type IgG isotypes in this group. FIG. 2c also shows that the percentage of CD8⁺ IFN-γ⁺ T cells tended to increase but did not reach significance.

FIG. 2d shows that following blood-stage malaria challenge all of the mice immunized with the AdM42-C4bp regime were protected. Table 1b shows that ten out of the 17 mice challenged displayed sterile immunity (as defined by the absence of patent parasitemia over the following 30 days), compared to 0/17 mice in the AdM42 group. Hence, AdM42-C4bp immunization not only provides complete protection against blood-stage challenge, but also provides a higher quality of protection as defined by the reduced levels of blood-stage parasitemia.

In Vivo Depletion of T Cells at the Time of pRBC Challenge does not Ablate Vaccine-Induced Protection.

To assess the protective contribution of vaccine-induced T cell responses in this model, mice were immunized as before with the most protective regime (AdM42-C4bp) and depleted of CD8⁺ or CD4⁺ T cells prior to pRBC challenge. Flow cytometric analysis indicated in vivo depletion to be >99% effective (data not shown). Table 1c shows depletion of T cells did not affect protective efficacy. Vaccine-induced antibody responses against MSP-1₄₂ are thus sufficient to completely protect mice in this model at the time of challenge.

Protection Against P. yoelii pRBC Challenge Correlates with the Level of Vaccine-Induced MSP-1₁₉-Specific IgG2a.

We had previously detected increased antigen-specific IgG induction following immunization with increased AdHu5 vaccine dose (data unpublished). To investigate this with respect to MVA, BALB/c mice were primed with AdHu5 and boosted using either 10⁶ pfu or 5×10⁷ pfu MVA. FIG. 3a shows that in the case of both vaccine constructs, the mice which were boosted with a lower dose of MVA developed significantly lower MSP-1₁₉-specific whole IgG responses. However, a different hierarchy was apparent following IgG isotype analysis. FIG. 3b shows that as seen before, immunization with vectors expressing MSP-1₄₂—C4bp induced more Th1-type IgG. Therefore, despite a relatively lower level of whole IgG in mice immunized with AdM42-C4bp (using 10⁶ pfu MVA), they possessed a significantly higher level of IgG2a when compared to mice immunized with AdM42 and boosted with either dose of MVA as seen in FIG. 3b.

FIG. 3c shows that following AdM42 immunization using 10⁶ pfu MVA to boost, only 40% of mice survived challenge with P. yoelii pRBCs. However, all of the mice survived in the AdM42-C4bp group and one third of these displayed sterile immunity, despite using the lower dose of MVA as seen from FIG. 3d and Table 1d. Consequently, fusion of mC4bp to MSP-1₄₂ also maintains enhanced protective efficacy of AdM vaccination at lower vaccine dose. Analysis of the mean levels of whole IgG, IgG1 or IgG2a versus % protection for all groups challenged showed there was a significant correlation between % survival and MSP-1₁₉-specific IgG2a (Spearman r=0.928, p=0.017), but not with whole IgG or IgG1.

AdHu5-MVA Immunization can Protect Against P. yoelii Sporozoite Challenge and Against the Liver-Stage Exo-Erythrocytic Forms.

Malaria challenge with pRBCs does not mimic the normal route of malaria transmission by the mosquito vector. The efficacy of AdHu5-MVA immunization against P. yoelii sporozoite challenge was thus investigated.

BALB/c mice were immunized as before with AdM42 or AdM42-C4bp (boosting with 5×10⁷ pfu MVA). Two weeks post-boost, mice were challenged with 50 P. yoelii sporozoites. Table 1e shows all of the mice developed patent blood-stage parasitemia, bar one mouse in the AdM42-C4bp group, but as seen from FIG. 4a-b these were all able to control blood-stage parasite growth and clear the infection. Similar to that observed in the pRBC challenge model, mice immunized with AdM42-C4bp displayed [a better quality of blood-stage immunity] lower parasite densities compared to those mice immunized with AdM42 as seen in Table 1a-b, e. Table 1e shows that naïve mice succumbed to blood-stage infection in an identical manner to those challenged with 10⁴ pRBCs.

Given the induction of blood-stage immunity by these vaccines, it is not possible to tell from these data whether a reduction in blood-stage parasitemia following sporozoite challenge is also partly due to a reduction in the liver-stage parasite burden. These vaccination regimes elicit strong T cell responses against MSP-1₃₃ see FIG. 2c, so it is possible that these effector T cells can protect against the late exo-erythrocytic schizonts¹⁹. To address this, mice were immunized and challenged with 5,000 P. yoelii sporozoites. An established real-time RT-PCR assay³² was used to quantify the liver-stage parasite burden 48 hours after challenge, FIG. 4c shows that there is a significant reduction in mice immunized with both vaccine regimes. These data are in agreement with the previous challenge study, shown in FIGS. 4a and 4b whereby a mean reduction in liver-stage parasite burden by 35-40% would not result in sterile immunity and hence not prevent blood-stage infection. Hence the induction of T cell and antibody responses against MSP-1 following AdHu5-MVA immunization can provide multi-stage protection against malaria infection.

AdM42-C4bp Immunization can Protect Against P. yoelii Liver- and Blood-Stage Parasites in C57BL/6 Mice.

Protection against both the liver- and blood-stage parasites of P. yoelii is reported to be under genetic control^{40,41}. To assess whether this novel immunization strategy could also protect mice on a differing genetic background, we assessed efficacy in C57BL/6 mice. Mice were immunized with the same AdM42 or AdM42-C4bp regimes using 5×10⁷ pfu MVA to boost. Assessment of immune responses indicated a different pattern of immunogenicity to that seen in BALB/c mice. As shown in FIG. 5a whole IgG responses against MSP-1₁₉ and MSP-1₃₃ showed a marginal, non-significant increase in the AdM42-C4bp group. MSP-1₃₃-specific CD8⁺ IFN-γ⁺ T cell responses could be measured in the spleen and, as seen from FIG. 5b unlike BALB/c, CD4⁺ IFN-γ⁺ T cell responses could be detected against both MSP-1₃₃ and MSP-1₁₉. FIG. 5b also shows that the CD4⁺ IFN-γ⁺ T cell response against MSP-1₃₃, but not MSP-1₁₉, was significantly enhanced when using the MSP-1₄₂—C4bp construct, and the CD8⁺ IFN-γ⁺ T cell response to MSP-1₃₃ was enhanced three-fold. As shown in Table 1f when groups of mice were challenged with 10⁴ pRBCs, all of the immunized mice were protected, with those mice in the AdM42-C4bp group again showing a higher degree of protection compared to the AdM42 group. Additionally as shown in FIG. 5c a significant reduction in liver-stage parasite burden was observed in the AdM42-C4bp group following challenge with 5,000 sporozoites, correlating with the results from FIG. 5b showing enhanced CD8⁺ IFN-γ⁺ T cell response in this group. These data indicate that the immunogenicity and protective efficacy of AdM42-C4bp immunization is not restricted to one genetic background, and that an equivalent strategy targeting P. falciparum MSP-1 may thus be efficacious in a genetically diverse human population.

### Discussion

This report describes the first use of replication-deficient adenovirus and MVA vaccine vectors to target blood-stage malaria, specifically MSP-1₄₂ in a prime-boost immunisation regime. This immunization regime is highly immunogenic not only for MSP-1₄₂-specific cellular responses but also surprisingly immunogenic for antibody induction. We have found AdHu5-MVA is by far the best regime for the induction of antibody responses when compared to homologous prime-boost, or other heterologous prime-boost regimes such as DNA-MVA, MVA-AdHu5 and DNA-AdHu5 (data unpublished). Following a single immunization with Ad42, antibody responses against MSP-1₁₉ continued to rise over a two month period, similar to that reported for other recombinant AdHu5 vaccines^{35,42}. Administration of the MVA boost after eight weeks rather than two led to enhanced antibody levels, thus reaching the high protective threshold required to protect against blood-stage P. yoelii. AdHu5 vaccines have shown strong immunogenicity in other studies, and this prolonged period may be required for the formation of optimal B cell and T helper cell memory populations, which are more effectively boosted by MVA. Similar findings on a long inter-vector interval were reported when using AdHu5 and live-replication competent vaccinia virus to target P. yoelii circumsporozoite protein³⁶.

Enhanced protective efficacy was achieved against blood-stage challenge in both BALB/c and C57BL/6 mice when AdHu5-MVA vectors expressing MSP-1₄₂ fused to mC4bp were used. This structural motif has been shown to adjuvant humoral immune responses to recombinant MSP-1₁₉ [WO2005/014654]. Here we report the ability of this motif to not only adjuvant MSP-1-specific antibody responses and CD4⁺ IFN-γ⁺ T cell responses in BALB/c mice, but also CD8⁺ and CD4⁺ IFN-γ⁺ T cell responses in C57BL/6 mice. The mechanism by which this occurs remains unknown, and may include oligomerization of the antigen⁴³, or the ability of this core domain to bind CD40⁴⁴ or C-reactive protein (CRP)⁴⁵. The latter two may effectively target antigens fused to mC4bp to receptors on antigen-presenting cells, although notably CRP is a minor acute-phase protein in mice.

This study did not assess whether antibodies against MSP-1₃₃ played a role in blood-stage immunity, however, they have been reported not to protect against P. yoelii¹⁰. The generation of AdHu5 and MVA vectors expressing MSP-1₃₃ or MSP-1₁₉ will answer this question. Nevertheless, the levels of MSP-1₁₉-specific IgG2a strongly correlated with enhanced blood-stage immunity. This is in agreement with other studies in the P. yoelii model, for example those using CpG⁴⁶ or IL-12⁴⁷ vaccine adjuvants, and for vaccines targeting P. falciparum apical membrane antigen-1⁴⁸. Th1-type isotypes (IgG1 and IgG3) are also associated with naturally-acquired immunity in humans⁴⁹. Th2-type IgG1, induced to very high titre by classic adjuvants such as Freund's, also correlates with protection⁹. However following just two immunizations and moderate levels of IgG induction, Th1-type IgG isotypes may provide more effective protection than Th2-type isotypes.

CD4⁺ T cell depletion did not abolish blood-stage immunity in these mice. CD4⁺ T cell lines specific to MSP-1₃₃ have been reported to protect mice against P. yoelii following adoptive transfer¹⁵, although other studies using physiologically-relevant vaccination regimes and CD4⁺ T cell depletion have shown either no loss or a variable reduction in immunity^{9,46}. Despite the description of CD4⁺ T cell epitopes in P. yoelii MSP-1₁₉¹⁴, we only detected CD4⁺ IFN-γ⁺ T cell responses to this domain in C57BL/6 mice. Previous studies have described the refractory nature of MSP-1₁₉ to antigen-processing¹³, and so it remains likely that the CD4⁺ T helper cell response against MSP-1₃₃ is important for antibody responses against MSP-1₁₉. CD8⁺ IFN-γ⁺ T cell responses were only detected against MSP-1₃₃. Their ability to mediate liver-stage immunity is in agreement with another study which demonstrated a comparable partial reduction in the liver-stage burden¹⁹, and argues for the use of MSP-1₄₂-rather than MSP-1₁₉-based vaccines.

These findings offer a new vaccination strategy that can induce protective immunity against blood-stage malaria on a par with multiple doses of recombinant protein-in-adjuvant formulations^{9,10}. However, unlike these protein vaccines, the induction of strong T cell responses to a blood-stage antigen using these vectors can also provide partial liver-stage immunity, and should allow for combination of these with partially effective pre-erythrocytic antigen vectors. To our knowledge, this is the first demonstration that complete multi-stage immunity against malaria can be induced in an animal model with only two immunizations or using viral vector vaccines. This successful vaccination strategy should also be applicable to the numerous pathogens where both strong T cells and antibodies are required for protection.

The enhancement of T cell immunogenicity with a simple adjuvantation strategy has been a long sought goal of vaccinology. We report here the induction of substantially enhanced CD4 and CD8 T cell responses by simply adding a extension to the antigen gene in a vector to encode the C4bp core domain. It is envisaged that appropriate adjustment of the sequence might be required to achieve optimal enhancement of immunogenicity in different species, for example by matching the C4bp sequence core sequence to the sequence of the vaccine. C4bp core sequences from various species are known or are readily determined by molecular biological methods.

TABLE 1

| | | | | |
|---|---|---|---|---|
| Immunization | | No. | | |
| Regime (Prime- | | Protected/ | | Median (Range) Peak % |
| Boost | Dose | No. | % | Parasitemia of |
| Interval) | MVA | Challenged | Protection | Protected Mice |

(a) AdHu5-MVA-MSP-1₄₂ in BALB/c mice (pRBC challenge).

| | | | | |
|---|---|---|---|---|
| AdM42 (2 wk) | 5 × 10⁷ | 0/6 | 0% | N/A |
| | pfu | | | |
| AdM42 (8 wk) | 5 × 10⁷ | 4/5 + 4/6 + | 76% | 1.2% (0.004%-27.7%) |
| | pfu | 5/6 | | |
| Naïve | N/A | 0/40 | 0% | N/A |
| Unimmunized | | | | |

(b) AdHu5-MVA MSP-1₄₂-C4bp in BALB/c mice (pRBC challenge).

| | | | | |
|---|---|---|---|---|
| AdM42-C4bp (8 wk) | 5 × 10⁷ | 6/6 + 11/11 | 100% | 0% (0%-9.1%) † |
| | pfu | | | |

(c) In vivo T cell depletion in AdM42-C4bp (8 wk) immunized BALB/c
mice (pRBC challenge).

| | | | | |
|---|---|---|---|---|
| No depletion | 5 × 10⁷ | 5/5 | 100% | 0.008% (0%-2.4%) |
| | pfu | | | |
| CD4⁺in vivo | 5 × 10⁷ | 5/5 | 100% | 0.008% (0%-0.95%) |
| Depletion | pfu | | | |
| CD8⁺in vivo | 5 × 10⁷ | 5/5 | 100% | 0% (0%-0.26%) |
| Depletion | pfu | | | |

(d) Low dose MVA boost (10⁶ pfu) in BALB/c mice (pRBC challenge).

| | | | | |
|---|---|---|---|---|
| AdM42 (8 wk) | 10⁶ pfu | 2/5 | 40% | 7.8% (3.8%-11.8%) |
| AdM42-C4bp (8 wk) | 10⁶ pfu | 6/6 | 100% | 0.042% (0%-5.2%)†† |

(e) BALB/c mice (sporozoite challenge).

| | | | | |
|---|---|---|---|---|
| AdM42 (8 wk) | 5 × 10⁷ | 6/6 | 100% | 17.3% (4.6%-31.8%) |
| | pfu | | | |
| AdM42-C4bp (8 wk) | 5 × 10⁷ | 6/6 | 100% | 1.1% (0%-6.1%)††† |
| | pfu | | | |
| Naïve | N/A | 0/6 | 0% | N/A |
| Unimmunized | | | | |

(f) C57BL/6 mice (pRBC challenge).

| | | | | |
|---|---|---|---|---|
| AdM42 (8 wk) | 5 × 10⁷ | 6/6 | 100% | 14.7% (3.7%-56.4%) |
| | pfu | | | |
| AdM42-C4bp (8 wk) | 5 × 10⁷ | 6/6 | 100% | 7.7% (2.2%-13.8%) |
| | pfu | | | |
| Naïve | N/A | 0/6 | 0% | N/A |
| Unimmunized | | | | |

Table 1. shows the protection against P. yoelii malaria challenge provided by immunisation with various vaccines. Mice were immunized and challenged with either 10⁴ P. yoelii pRBCs or 50 P. yoelii sporozoites as described. The strain of mouse, immunization regime employed, dose of MVA and numbers protected are indicated. In protected groups, the median and range of peak blood-stage % parasitemias are indicated for those mice which survived the challenge. † 10/17 mice in this group showed sterile immunity (as defined by the absence of patent parasitemia over the 30 day period following challenge). †† 2/6 and ††† ⅙ mice in these groups showed sterile immunity.

### REFERENCES

1. Snow, R. W., Guerra, C. A., Noor, A. M., Myint, H. Y. & Hay, S. I. The global distribution of clinical episodes of Plasmodium falciparum malaria. Nature 434, 214-7 (2005).
2. Greenwood, B. M., Bojang, K., Whitty, C. J. & Targett, G. A. Malaria. Lancet 365, 1487-98 (2005).
3. Riley, E. M. et al. Naturally acquired cellular and humoral immune responses to the major merozoite surface antigen (PfMSP1) of Plasmodium falciparum are associated with reduced malaria morbidity. Parasite Immunol 14, 321-37 (1992).
4. Mahanty, S., Saul, A. & Miller, L. H. Progress in the development of recombinant and synthetic blood-stage malaria vaccines. J Exp Biol 206, 3781-8 (2003).
5. Holder, A. A. et al. Processing of the precursor to the major merozoite surface antigens of Plasmodium falciparum. Parasitology 94 (Pt 2), 199-208 (1987).
6. Blackman, M. J., Heidrich, H. G., Donachie, S., McBride, J. S. & Holder, A. A. A single fragment of a malaria merozoite surface protein remains on the parasite during red cell invasion and is the target of invasion-inhibiting antibodies. J Exp Med 172, 379-82 (1990).
7. Tian, J. H., Kumar, S., Kaslow, D. C. & Miller, L. H. Comparison of protection induced by immunization with recombinant proteins from different regions of merozoite surface protein 1 of Plasmodium yoelii. Infect Immun 65, 3032-6 (1997).
8. Darko, C. A. et al. The clinical-grade 42-kilodalton fragment of merozoite surface protein 1 of Plasmodium falciparum strain FVO expressed in Escherichia coli protects Aotus nancymai against challenge with homologous erythrocytic-stage parasites. Infect Immun 73, 287-97 (2005).
9. Hirunpetcharat, C. et al. Complete protective immunity induced in mice by immunization with the 19-kilodalton carboxyl-terminal fragment of the merozoite surface protein-1 (MSP1[19]) of Plasmodium yoelii expressed in Saccharomyces cerevisiae: correlation of protection with antigen-specific antibody titer, but not with effector CD4+ T cells. J Immunol 159, 3400-11 (1997).
10. Ahlborg, N., Ling, I. T., Howard, W., Holder, A. A. & Riley, E. M. Protective immune responses to the 42-kilodalton (kDa) region of Plasmodium yoelii merozoite surface protein 1 are induced by the C-terminal 19-kDa region but not by the adjacent 33-kDa region. Infect Immun 70, 820-5 (2002).
11. Yuen, D. et al. Antigenicity and immunogenicity of the N-terminal 33-kDa processing fragment of the Plasmodium falciparum merozoite surface protein 1, MSP1: implications for vaccine development. Vaccine 25, 490-9 (2007).
12. Good, M. F., Xu, H., Wykes, M. & Engwerda, C. R. Development and regulation of cell-mediated immune responses to the blood stages of malaria: implications for vaccine research. Annu Rev Immunol 23, 69-99 (2005).
13. Hensmann, M. et al. Disulfide bonds in merozoite surface protein 1 of the malaria parasite impede efficient antigen processing and affect the in vivo antibody response. Eur J Immunol 34, 639-48 (2004).
14. Tian, J. H. et al. Definition of T cell epitopes within the 19 kDa carboxylterminal fragment of Plasmodium yoelii merozoite surface protein 1 (MSP1(19)) and their role in immunity to malaria. Parasite Immunol 20, 263-78 (1998).
15. Wipasa, J. et al. Identification of T cell epitopes on the 33-kDa fragment of Plasmodium yoelii merozoite surface protein 1 and their antibody-independent protective role in immunity to blood stage malaria. J Immunol 169, 944-51 (2002).
16. Elliott, S. R., Kuns, R. D. & Good, M. F. Heterologous immunity in the absence of variant-specific antibodies after exposure to subpatent infection with blood-stage malaria. Infect Immun 73, 2478-85 (2005).
17. Pombo, D. J. et al. Immunity to malaria after administration of ultra-low doses of red cells infected with Plasmodium falciparum. Lancet 360, 610-7 (2002).
18. Vinetz, J. M. et al. Adoptive transfer of CD8+ T cells from immune animals does not transfer immunity to blood stage Plasmodium yoelii malaria. J Immunol 144, 1069-74 (1990).
19. Kawabata, Y. et al. Merozoite surface protein 1-specific immune response is protective against exoerythrocytic forms of Plasmodium yoelii. Infect Immun 70, 6075-82 (2002).
20. Sacci, J. B., Jr. et al. Transcriptional analysis of in vivo Plasmodium yoelii liver stage gene expression. Mol Biochem Parasitol 142, 177-83 (2005).
21. Dunachie, S. J. & Hill, A. V. Prime-boost strategies for malaria vaccine development. J Exp Biol 206, 3771-9 (2003).
22. Bejon, P. et al. Safety profile of the viral vectors of attenuated fowlpox strain FP9 and modified vaccinia virus Ankara recombinant for either of 2 preerythrocytic malaria antigens, ME-TRAP or the circumsporozoite protein, in children and adults in Kenya. Clin Infect Dis 42, 1102-10 (2006).
23. Catanzaro, A. T. et al. Phase 1 safety and immunogenicity evaluation of a multiclade HIV-1 candidate vaccine delivered by a replication-defective recombinant adenovirus vector. J Infect Dis 194, 1638-49 (2006).
24. Gilbert, S. C. et al. Enhanced CD8 T cell immunogenicity and protective efficacy in a mouse malaria model using a recombinant adenoviral vaccine in heterologous prime-boost immunisation regimes. Vaccine 20, 1039-45 (2002).
25. Reyes-Sandoval, A. et al. Single dose immunogenicity and protective efficacy of simian adenoviral vectors against P. berghei. Submitted (2007).
26. Blom, A. M., Villoutreix, B. O. & Dahlback, B. Complement inhibitor C4b-binding protein-friend or foe in the innate immune system? Mol Immunol 40, 1333-46 (2004).
27. Lewis, A. P. Cloning and analysis of the gene encoding the 230-kilodalton merozoite surface antigen of Plasmodium yoelii. Mol Biochem Parasitol 36, 271-82 (1989).
28. McShane, H., Behboudi, S., Goonetilleke, N., Brookes, R. & Hill, A. V. Protective immunity against Mycobacterium tuberculosis induced by dendritic cells pulsed with both CD8(+)- and CD4(+)-T-cell epitopes from antigen 85A. Infect Immun 70, 1623-6 (2002).
29. McShane, H., Brookes, R., Gilbert, S. C. & Hill, A. V. Enhanced immunogenicity of CD4(+) t-cell responses and protective efficacy of a DNA-modified vaccinia virus Ankara prime-boost vaccination regimen for murine tuberculosis. Infect Immun 69, 681-6 (2001).
30. Hutchings, C. L., Gilbert, S. C., Hill, A. V. & Moore, A. C. Novel protein and poxvirus-based vaccine combinations for simultaneous induction of humoral and cell-mediated immunity. J Immunol 175, 599-606 (2005).
31. Briones, M. R., Tsuji, M. & Nussenzweig, V. The large difference in infectivity for mice of Plasmodium berghei and Plasmodium yoelii sporozoites cannot be correlated with their ability to enter into hepatocytes. Mol Biochem Parasitol 77, 7-17 (1996).
32. Bruna-Romero, O. et al. Detection of malaria liver-stages in mice infected through the bite of a single Anopheles mosquito using a highly sensitive real-time PCR. Int J Parasitol 31, 1499-502 (2001).
33. McShane, H. et al. Recombinant modified vaccinia virus Ankara expressing antigen 85A boosts BCG-primed and naturally acquired antimycobacterial immunity in humans. Nat Med 10, 1240-4 (2004).
34. Chen, Z. et al. Recombinant modified vaccinia virus Ankara expressing the spike glycoprotein of severe acute respiratory syndrome coronavirus induces protective neutralizing antibodies primarily targeting the receptor binding region. J Virol 79, 2678-88 (2005).
35. Sullivan, N. J. et al. Accelerated vaccination for Ebola virus haemorrhagic fever in non-human primates. Nature 424, 681-4 (2003).
36. Bruna-Romero, O., Gonzalez-Aseguinolaza, G., Hafalla, J. C., Tsuji, M. & Nussenzweig, R. S. Complete, long-lasting protection against malaria of mice primed and boosted with two distinct viral vectors expressing the same plasmodial antigen. Proc Natl Acad Sci USA 98, 11491-6 (2001).
37. Dempsey, P. W., Allison, M. E., Akkaraju, S., Goodnow, C. C. & Fearon, D. T. C3d of complement as a molecular adjuvant: bridging innate and acquired immunity. Science 271, 348-50 (1996).
38. Ross, T. M., Xu, Y., Bright, R. A. & Robinson, H. L. C3d enhancement of antibodies to hemagglutinin accelerates protection against influenza virus challenge. Nat Immunol 1, 127-31 (2000).
39. Doolan, D. L. & Hoffman, S. L. The complexity of protective immunity against liver-stage malaria. J Immunol 165, 1453-62 (2000).
40. Tian, J. H. et al. Genetic regulation of protective immune response in congenic strains of mice vaccinated with a subunit malaria vaccine. J Immunol 157, 1176-83 (1996).
41. Weiss, W. R., Good, M. F., Hollingdale, M. R., Miller, L. H. & Berzofsky, J. A. Genetic control of immunity to Plasmodium yoelii sporozoites. J Immunol 143, 4263-6 (1989).
42. Gao, W. et al. Protection of mice and poultry from lethal H5N1 avian influenza virus through adenovirus-based immunization. J Virol 80, 1959-64 (2006).
43. Christiansen, D. et al. Octamerization enables soluble CD46 receptor to neutralize measles virus in vitro and in vivo. J Virol 74, 4672-8 (2000).
44. Brodeur, S. R. et al. C4b-binding protein (C4BP) activates B cells through the CD40 receptor. Immunity 18, 837-48 (2003).
45. Sjoberg, A. P., Trouw, L. A., McGrath, F. D., Hack, C. E. & Blom, A. M. Regulation of complement activation by C-reactive protein: targeting of the inhibitory activity of C4b-binding protein. J Immunol 176, 7612-20 (2006).
46. Hirunpetcharat, C. et al. CpG oligodeoxynucleotide enhances immunity against blood-stage malaria infection in mice parenterally immunized with a yeast-expressed 19 kDa carboxyl-terminal fragment of Plasmodium yoelii merozoite surface protein-1 (MSP1(19)) formulated in oil-based Montanides. Vaccine 21, 2923-32 (2003).
47. Sakai, T. et al. Gene gun-based co-immunization of merozoite surface protein-1 cDNA with IL-12 expression plasmid confers protection against lethal Plasmodium yoelii in A/J mice. Vaccine 21, 1432-44 (2003).
48. Mullen, G. E. et al. Enhancement of functional antibody responses to AMA1-C1/Alhydrogel, a Plasmodium falciparum malaria vaccine, with CpG oligodeoxynucleotide. Vaccine 24, 2497-505 (2006).
49. Bouharoun-Tayoun, H. & Druilhe, P. Plasmodium falciparum malaria: evidence for an isotype imbalance which may be responsible for delayed acquisition of protective immunity. Infect Immun 60, 1473-81 (1992).

1. An immunogenic composition comprising a viral vector, said vector comprising a nucleic acid sequence encoding a C4bp domain or variant or fragment thereof and a nucleic acid sequence encoding an antigen of interest.

2. The immunogenic composition according to claim 1, wherein the nucleic acid encoding the C4bp domain is in frame with the nucleic acid encoding the antigen of interest.

3. The immunogenic composition according to claim 1, wherein the encoded C4bp domain is selected from the group consisting of the amino acid sequences shown in FIG. 6 or FIG. 7.

4. The immunogenic composition according to claim 1, wherein the C4bp domain and antigen of interest are expressed as a fusion protein.

5. The immunogenic composition according to claim 1, wherein the viral vector is selected from the group consisting of a poxvirus vector such as modified vaccinia virus Ankara (MVA), an avipox vector such as a fowlpox, canarypox or ALVAC, an herpesvirus vector (including herpes simplex and CMV), an alphavirus vector and an adenovirus vector.

6. The immunogenic composition according to claim 1, wherein the viral vector is a pox virus vector or an adenovirus vector.

7. The immunogenic composition according to claim 1, wherein the antigen is a malaria antigen.

8. The immunogenic composition according to claim 7, wherein the antigen is ME-TRAP, CSP, MSP-1 or fragments thereof, or AMA1.

9. The immunogenic composition according to claim 7, wherein the antigen is a blood-stage malarial antigen.

10. The immunogenic composition of claim 1 admixed with one or more pharmaceutically acceptable vehicles, carriers, diluents, or adjuvants.

11. A vaccine comprising the immunogenic composition of claim 1.

12. A product, combination or kit comprising;
a) a priming composition comprising a first viral vector, said viral vector further comprising a nucleic acid encoding a C4bp domain or variant of fragment thereof, and at least one pathogen or tumour antigen; and
b) a boosting composition comprising a second viral vector, said second viral vector being different from said first viral vector and further comprising a nucleic acid encoding a C4bp domain, and at least one pathogen or tumour antigen which is the same as the pathogen or tumour antigen of the priming composition,

13. The product, combination or kit according to claim 12, wherein the nucleic acid encoding the C4bp domain is in frame with the nucleic acid encoding the antigen of interest.

14. The product, combination or kit according to claim 12, wherein the encoded C4bp domain is selected from the group consisting of the amino acid sequences shown in FIG. 6 or FIG. 7.

15. The product, combination or kit according to claim 12, wherein the C4bp domain and antigen of interest in each vector are expressed as a fusion protein.

16. The product, combination or kit according to claim 12, wherein each viral vector is selected from the group consisting of a poxvirus vector such as modified vaccinia virus Ankara (MVA), an avipox vector such as a fowlpox, canarypox or ALVAC, an herpesvirus vector (including herpes simplex and CMV), an alphavirus vector and an adenovirus vector.

17. The product, combination or kit according to claim 12, wherein said first viral vector is an adenoviral vector.

18. The product, combination or kit according to claim 12, wherein said second viral vector is a pox virus vector.

19. The product, combination or kit according to claim 12, wherein the antigen is a malaria antigen.

20. A viral vector comprising a nucleic acid sequence encoding a C4bp domain or variant or fragment thereof and a nucleic acid encoding the antigen of interest.

21. The viral vector according to claim 20, wherein the nucleic acid encoding the C4bp domain is in frame with the nucleic acid encoding the antigen of interest.

22. The viral vector according to claim 20, wherein the encoded C4bp domain is selected from the group consisting of the amino acid sequences shown in FIG. 6 or FIG. 7.

23. The viral vector according to claim 20, wherein the C4bp domain and antigen of interest are expressed as a fusion protein.

24. The viral vector according to claim 20, wherein the viral vector is selected from the group consisting of a poxvirus vector such as modified vaccinia virus Ankara (MVA), an avipox vector such as a fowlpox, canarypox or ALVAC, an herpesvirus vector (including herpes simplex and CMV), an alphavirus vector and an adenovirus vector.

25. The viral vector according to claim 20, wherein the viral vector is a pox virus vector or an adenovirus vector.

26. The viral vector according to claim 20, wherein the antigen is a malaria antigen.

27.-29. (canceled)

30. A method of immunising an individual by administering an effective amount of at least one immunogenic composition according to claim 1.

31. The method according to claim 30, wherein the individual is immunised using a heterologous prime-boost regimen comprising administering to an individual in need thereof a first unit dose of a first immunogenic composition or vaccine and a second unit dose of a second immunogenic composition or vaccine.

32. The method according to claim 31, wherein the first immunogenic composition or vaccine forming the first unit dose comprises an adenoviral vector.

33. The method according to claim 31, wherein the second immunogenic composition or vaccine forming the second unit dose comprises a poxyiral vector.

34. The method according to claim 31, wherein the time period between administration of the first and second unit doses is 2-8 weeks.

35.-37. (canceled)
